(19) United States
(12) Patent Application Publication
(10) Pub. No.: US 2011/0160480 A1
(43) Pub. Date: Jun. 30, 2011

(54) Continuous Process for Obtaining a Lactic Ester

(75) Inventors: Delphine Hottois, Gussignies (FR); Alexandre Bruneau, Taintignies (Belgique) (BE); Jean-Christophe Bogaert, Ligne (BE); Philippe Coszach, Courcelles (BE)

(73) Assignee: GALACTIC SA, Escanaffles (BE)

(21) Appl. No.: 13/056,292

(22) PCT Filed: Jul. 16, 2009
(86) PCT No.: PCT/EP2009/059162

§ 371 Date: Mar. 11, 2011

(30) Foreign Application Priority Data
Jul. 30, 2008 (BE) 2008/0424

(51) Int. Cl. C07C 67/08

(52) U.S. Cl. 560/179

(57) ABSTRACT

Continuous method for obtaining a lactic ester, characterised in that it comprises the following steps:
a) esterification reaction of a composition comprising an alcohol in the presence of a composition comprising lactic acid in excess,
b) extraction of a vapour phase from this reaction medium, containing lactic ester, alcohol, water and traces of lactic acid;
c) distillation of the vapour phase obtained at step b) in order to recover the lactic ester, alcohol and water at the top;
d) distillation of the fraction obtained at step c) in order to recover the purified lactic ester at the bottom.

### FIELD OF THE INVENTION

The present invention concerns a continuous method for obtaining a purified lactic ester from a solution of lactic acid in excess and an alcohol.

### CONTEXT OF THE INVENTION

Lactic acids are hydroxylated esters, some being found naturally in small quantities in a wide variety of food, including wine, citrus fruit, etc. They are in liquid form, colourless, having a characteristic odour and are miscible with water and in many solvents. These lactic esters are solvents synthesised from removable materials and are consequently green solvents or biosolvents, commonly used in pharmaceutical preparations, as a food additive or as a flavouring. However, they are usually employed in combination with other solvents or in substitution for toxic solvents.

### PRIOR ART

The two optically active forms of lactic acid (L-LA) and (D-LA) can give a lactic ester in two stereoisomeric forms, L-lactic ester and D-lactic ester.

In the remainder of the document, the invention will be disclosed starting with lactic acid of the L(+) form but it could be applied to the other enantiomeric form or to the mixture of these. Lactic acid means any aqueous solution of lactic acid having a variable lactic acid purity and a concentration that may vary between 50% and 100% by weight and preferably between 80% and 100% by weight. It will be understood that these solutions are mixtures of water and monomers, dimers and trimers of lactic acid.

The present invention is applicable to various lactic esters and to the respective alcohols thereof, such as for example methanol in the case of methyl lactate or butanol for butyl lactate. However, in the remainder of the description, in order to simplify understanding, the invention will be made particular to the ester in ethyl lactate form.

The method of producing ethyl lactate most frequently used industrially starts from lactic acid and ethanol, which can also be obtained from renewable sources, via an esterification reaction as described in particular in the patents FR 2848208A1 and FR 2848209A1. This esterification between lactic acid and ethanol leads to the formation of water and ethyl lactate:

CH₃CHOHCOOH+C₂H₅OH [symbol] CH₃CHOHCOOC₂H₅+H₂O

The esterification reaction is a balanced chemical reaction, that is to say part of the water and ethyl lactate formed can react together in order to give lactic acid and the initial ethanol again. In this case it is a hydrolysis. Esterification reactions have the reputation of being athermal, that is to say the ΔᵣH° (298 K) thereof is close to zero.

The esterification reaction is catalysed by acids, whether they be mineral such as for example sulphuric acid, hydrochloric acid or para-toluene sulphonic acid, or organic (lactic acid therefore itself being able to fulfil the role of catalyst).

Because of the multifunctionality of lactic acid comprising a carboxylic function and an alcohol function, it is possible to observe successive intermolecular esterifications leading to the formation of oligomers:

CH₃CHOHCOOH+CH₃CHOHCOOH [symbol] CH₃CHOHCOOCH(CH₃)COOH+H₂O

According to the initial concentration of the monomeric lactic acid solutions, a certain equilibrium in distribution is established between the various oligomers. This oligomerisation reaction is a spontaneous reaction between two lactic acid molecules. On the other hand, in the presence of alcohol, this reaction is less or even non-existent. In this context and in order to eliminate these reactions, industrial processes take place in the presence of a large excess of ethanol in accordance for example with the patents FR 2848408A1 and FR 2848209A1 and generally the ethanol/lactic acid mol ratio is greater than 2:1.

Moreover, the fact that lactic acid oligomerises spontaneously may cause the formation of ethyl lactate oligomers during esterification. This is because, just like lactic acid, the various oligomers may react with ethanol in accordance with the reaction:

CH₃CHOHCOOCH(CH₃)COOH+C₂H₅OH→CH₃CHOHCOOCH(CH₃)COOC₂H₅+H₂O

Moreover, during the purification of ethyl lactate, the formation of ethyl lactate oligomers obtained by transesterification of two ester molecules may also be observed:

2CH₃CHOHCOOC₂H₅→CH₃CHOHCOOCH(CH₃)COOC₂H₅+C₂H₅OH

This reaction takes place during the heating of ethyl lactate during the purification step and is promoted by an acidic catalysis. In order to prevent this transesterification, it will be preferable to reduce the working temperature, for example by working under vacuum.

The production of ethyl lactate starting from lactic acid and ethanol is therefore not obvious to implement through:
1. the spontaneous formation of water following the various mechanisms stated above during esterification. However, the latter:
   causes, under certain conditions, hydrolysis of the ethyl lactate into lactic acid,
   causes the formation of an azeotrope between the ethanol and water, requiring an additional ethanol purification step,
   may cause the formation of a azeotrope between the ethyl lactate and water, complicating separation thereof,
2. the spontaneous formation of ethyl lactate oligomers during the esterification reaction complicating the purification of the ester.

### DETAILED DESCRIPTION OF THE INVENTION

In the remainder of the text, the percentages indicated will always be expressed by weight and the ratios will refer to mol ratios. Likewise, lactic acid and ethanol are considered to be pure or in aqueous solution.

The present invention overcomes these drawbacks by making it possible to obtain purified ethyl lactate by esterification of a lactic acid solution by ethanol. The method is characterised in that 1) the reaction is performed in the presence of an excess of lactic acid into which ethanol is introduced directly in order to effect an optimum catalysis, and the reaction is consequently auto-catalysed, 2) only the vapour phase is extracted continuously, composed of water, ethanol and ethyl lactate formed, as well as traces of lactic acid, at a temperature of between 110° and 140° C. and preferably between 115° and 125° C. under a vacuum of 15 to 40 kPa absolute. This gaseous mixture is injected approximately halfway up a first distillation column, working at the same reduced pressure, where it is separated into two fractions: a first collected at the head of the column and rich in water, ethanol and ethyl lactate, and a second mixture obtained at the foot of the column, rich in lactic acid. The fraction emerging at the head is then directed approximately halfway up a second distillation column, also working at reduced pressure, preferably less than the first column, in order to separate therein the ethyl lactate from the ethanol/water mixture.

More generally, the method is characterised in that it comprises the following steps:
a) esterification reaction of a composition comprising an alcohol in the presence of a composition comprising lactic acid in excess, the excess acid being characterised by a lactic acid/alcohol mol ratio of between 1.1:1 and 50:1;
b) extraction of a vapour phase from this reaction medium, containing lactic ester, alcohol, water and traces of lactic acid;
c) distillation of the vapour phase obtained at step b) in order to recover the lactic ester, alcohol and water at the top;
d) distillation of the fraction obtained at step c) in order to recover the purified lactic ester at the bottom.

The method described in the present invention is therefore characterised in that the ethanol can be replaced by an alcohol containing 1 to 12 carbons, such as methanol, ethanol, n-butanol, isobutanol, sec-butanol, tert-butanol, n-propanol, butanol, 2-ethylhexanol, 2-ethylbutanol or hexanol.

The purified lactic ester and in particular the purified ethyl lactate obtained by the method described in the invention corresponds to the conventional grade available on the market and is characterised by purity equal to or greater than 97%, a water content equal to or less than 0.3%, and acidity of less of than 0.1% and a colour less than 50 Hazen.

According to the present invention, the esterification reaction can be carried out at atmospheric pressure or under pressure, preferably between 15 and 300 kPa absolute, and at a temperature of between 110° and 200° C.

The esterification reactor is previously filled with a lactic acid charge and is heated to 100° C. The reaction consists, as from 100° C., of the continuous addition of a lactic acid solution from the top of the reactor and anhydrous or azeotropic ethanol, liquid or gaseous, from the bottom in order to disperse it throughout the reaction volume by virtue of the agitator. Any equipment for improving the dispersion of the ethanol in the medium (diffuser, spray or other) can be considered in the context of this invention. Likewise, any agitation profile for improving the dispersion of the ethanol in the medium (single-stage or multi-stage agitator, turbine of the “Rushton” type or other) can be considered in the context of this invention. The reactor will ideally have a geometry promoting the dispersion of the ethanol (increased by the liquid height) and vaporisation of the gaseous phase (increased by the diameter of the reactor). The use of anhydrous ethanol is preferable but not essential. The ethanol is characterised in that it contains at a minimum 30% ethanol. The ethanol/lactic acid mol ratio of what is fed into the esterification reactor is preferably between 1:1 and 5:1.

In the context of the present invention, part of the ethanol reacts with the lactic acid in order to produce ethyl lactate and water. It is also used as a stripping agent for promoting the extraction of volatiles from the reaction medium. It will however be ensured that this fraction is controlled in order to prevent a process that wastes too much energy through an excessive looping of ethanol.

The particularity of the present invention lies in the fact that:
although the reagents are supplied in such a way as to be in ethanol excess, through its stripping action, the esterification reaction takes place in an acid medium. Esterification is therefore made possible by the continuous extraction of the gaseous phase while limiting the entrainment of acidity,
it is not necessary to use a catalyst, since the catalysing agent is already in sufficient quantity in the reaction medium in the light of the excess of lactic acid, and the reaction is consequently auto-catalysed.

According to said method, the fraction collected at the top of the first column has a residual acidity of less than 0.5% (for 100% ethyl lactate), and preferably less than 0.2% in order to prevent oligomerisation of the ester, the reaction described previously. This acidity value can for example be obtained by adjusting the reflux level of the distillation column.

According to the invention, these condensates leaving at the top are then directed to a second distillation column, where they are injected approximately halfway up the column. This ethanol/water/ethyl lactate mixture is subjected to a distillation preferentially under reduced pressure, preferably less than or equal to 10 kPa absolute, from which an ethanol/water mixture is recovered at the top of the column and the purified ethyl lactate at the bottom; distillation carried out so as to reduce as far as possible the residence time of the mixture. The method of the invention produces a purified ethyl lactate.

The ethanol-water phase drawn off at the head of the second distillation column may, according to one embodiment, be separated in a distillation column functioning under reduced pressure or at atmospheric pressure according to the composition of the ethanol required. It may also be treated by an azeotropic distillation method.

According to another embodiment of the invention, the dehydration of the ethanol-water mixture may be carried out by means of the PSA (Pressure Switch Adsorption) technique, which consists of effecting the selective adsorption of water on a molecular sieve bed by making the azeotropic mixture pass therethrough. The anhydrous ethanol recovered is advantageously recycled in the reaction medium.

The ethanol-water mixture, according to another embodiment of the invention, may be treated by pervaporation. This is a method of separating the constituents by partial vaporisation through a dense membrane having a preferential affinity for one of the constituents.

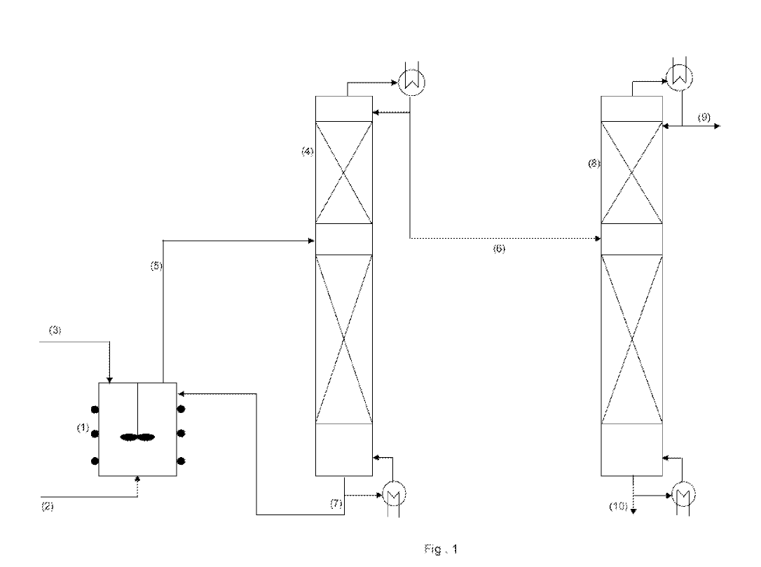

FIG. 1 describes schematically a particular embodiment of the invention. This device comprises:
a reactor (1) provided with an agitator and optionally counter blades, a temperature sensor, an ethanol supply (2) and a lactic acid supply (3);
a first distillation column (4) with lining, boiler, condenser and reflex system supplied in gaseous form via the supply pipe (5) coming from the reactor, provided with a head outlet (6) for the ethanol-water-ethyl lactate mixture and a bottom outlet (7) for the ethyl lactate containing lactic acid;
a second distillation column (8) with lining also surmounted by a boiler, a condenser and a reflux system supplied with liquid phase by the top condensates of the first column via the supply pipe (6), provided with a top outlet (9) for the ethanol-water mixture and an outlet (10) containing the purified ethyl lactate.

The following examples illustrate the present invention.

### EXAMPLES

### Example 1

A charge consisting of 500 kg of lactic acid with a 100% concentration by weight is first introduced into the esterification reactor with a capacity of 9 m³. The product is agitated and heated at 100° C. under a reduced pressure of 27.5 kPa absolute.

When the lactic acid is at the correct temperature, 165.5 kg/h of anhydrous ethanol and 100 kg/h of 100% lactic acid is introduced continuously, equivalent to an ethanol/lactic acid mol ratio of 3.6:1. Esterification takes place at 130° C., at a reduced pressure of 27.5 kPa absolute.

Part of the ethanol reacts with the lactic acid in order to produce ethyl lactate and water, and the other part is used as a stripping agent for promoting extraction of the volatiles from the reaction medium. This therefore means that the esterification reaction takes place in an excess of lactic acid in the reactor.

The volatile phase comprising water, ethanol, ethyl lactate and traces of lactic acid is extracted continuously from the reaction medium. The temperature of these vapours is 120° C. This mixture has an acidity of less than or equal to 0.2% (equivalent to 100% ethyl lactate).

This gaseous phase is injected continuously into a first distillation column, where it undergoes distillation under reduced pressure (27.5 kPa absolute). The column works at a reflux level of 2. A first fraction collected at the head, at a temperature of 70° C., is composed of:

47% ethanol,

21% water,

32% ethyl lactate.

The fraction at the bottom of the column is collected at a temperature of 137° C. and consists of 10% lactic acid and 90% ethyl lactate. This mixture is recycled to the esterification reactor.

The fraction collected at the top of the first column is then injected in liquid form into a second distillation column in order to purify the ester. This column works at a pressure of 10 kPa absolute and a reflux level of 0.2. The purified ester is collected at the bottom of the column at a temperature of 82° C. and is composed of ethyl lactate with a purity greater than 97%. The ethanol/water mixture is recovered at the top of the column at a temperature of 30° C.

The change in composition of the ethyl lactate collected in the boiler at the bottom of the second column is set out in the following table:

Phase of starting columns and being brought up to full operation

| | | | | | | Ethyl |
|---|---|---|---|---|---|---|
| | | Water | | | Ethanol | lactate |
| | | content | Acidity | Colour | content | content |
| Time | | (%) | (%) | (Hazen) | (%) | (%) |
| 5 | hours | 0.12 | 0.04 | 5 | 0.2 | 99.64 |
| 8 | hours | 0.08 | 0.02 | 13 | 0.1 | 99.88 |
| 10 | hours | 0.06 | 0.05 | 9 | 0.5 | 99.39 |
| 24 | hours | 0.1 | 0.06 | 5 | 0.3 | 99.54 |
| 2 | days | 0.08 | 0.01 | 12 | 0.1 | 99.81 |
| 3 | days (*) | 0.05 | 0.07 | 9 | 0.4 | 99.48 |
| 3 | days | 0.05 | 0.09 | 18 | 0.4 | 99.46 |
| 4 | days | 0.11 | 0.05 | 11 | 0.2 | 99.64 |

(*) Sample taken off directly at the lining output rather than in the boiler.

### Example 2

In this example, the method disclosed in example 1 is repeated with an ethanol containing 70% water.

The fraction collected at the top of the first column, at a temperature of 70° C., is in this case composed of:

14.1% ethanol,

69.9% water,

16% ethyl lactate.

It can therefore be seen that the method allows the use of an ethanol containing water but that the magnitude of the content thereof impacts on the productivity of ethyl lactate.

### Example 3

During tests in flasks for the batch distillation of an ethanol/water/ethyl lactate/lactic acid mixture, we found on several occasions that the distillation of ethanol and then water took place correctly but that, as soon as these disappeared, the distillation of the ethyl lactate did not function correctly. It is observed that the purity of the ethyl lactate obtained at the top decreases over time and that the amount of oligomer at the bottom of the column increases significantly.

We have assumed that the presence of lactic acid catalysed the oligomerisation reaction of the ethyl lactate and that the latter was appreciably amplified when the reaction medium was devoid of water and ethanol, the only compounds capable of hydrolysing or transesterifying the oligomers formed.

In order to verify this effect, we carried out several distillation experiments at atmospheric pressure on synthetic mixtures.

The experimental device consists of a 500 ml flask intended to receive the mixture to be distilled. The latter is inserted in a stirred flask heater, the heating power of which is adjusted to its maximum. The column is filled with a non-structured lining and is isolated from the ambient air. At the top of the column and in the flask two thermometers are arranged, for monitoring the change in temperature of the vapours and that of the mixture. The vapours are recovered in a water condenser. The condensates are recovered in a 250 ml flask and regularly weighed. The column used does not have any reflux.

The synthesis mixtures all initially contain 10% ethanol, 30% water and a minimum of 58% ethyl lactate according to the quantity of lactic acid introduced (2%, 1%, 0.5% and 0.2% by weight).

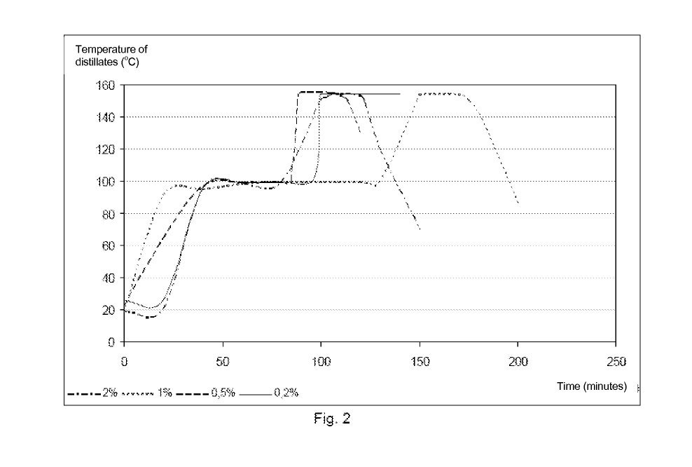

FIG. 2 shows the change in temperature of the vapours at the top of the column for each distillation test as a function of time.

We note that, though the first part of the separation takes place correctly (extraction of ethanol and water), the distillation of the ethyl lactate is more tricky. If we monitor the change in temperature at the top of the distillation column as a function of time, we find three temperature levels corresponding to the boiling points of pure ethanol and, very close, of the water/ethanol azeotrope (78° C.), pure water (100° C.) and pure ethyl lactate (154° C.). During these tests, we find that, for 15 minutes, the temperature at the top remains constant, close to 154° C., and then drops fairly rapidly.

On the other hand, for the mixture containing initially 0.2% lactic acid, this temperature at the head remains constant. It is the threshold value below which the presence of lactic acid no longer interferes with the distillation.

From these tests, we thus determined the maximum lactic acid content in the fraction collected at the head of the first distillation column, a content ideally less than or equal to 0.2%.

### Example 4

In this example, the procedure set out in example 1 is repeated in the case of methanol and an excess of lactic acid.

The result obtained after 24 hours and 3 days of operation are set out in the following table:

| | | | | | Methyl |
|---|---|---|---|---|---|
| | | Water | | | lactate |
| | | content | Acidity | Colour | content |
| Time | | (%) | (%) | (Hazen) | (%) |
| 24 | hours | 0.2 | 0.17 | 7 | 99.09 |
| 3 | days | 0.3 | 0.12 | 9 | 99.58 |

### Example 5

The procedure is similar to example 1 for the synthesis of butyl lactate starting from n-butanol and an excess of lactic acid.

The ester obtained after 24 hours of operation has a purity of 99.5%, an acidity of 0.05% and a water content of 0.25%.

### Example 6

The method set out in example 1 is reiterated in order to synthesise 2-ethylhexanol lactate.

The results obtained after several days of operation are set out in the following table:

| | | Water | | | 2-ethylhexanol |
|---|---|---|---|---|---|
| | | content | Acidity | Colour | lactate content |
| Time | | (%) | (%) | (Hazen) | (%) |
| 1 | day | 0.02 | 0.03 | 13 | 99.95 |
| 6 | days | 0.11 | 0.07 | 20 | 99.82 |

1. Continuous method for obtaining a lactic ester, characterised in that it comprises the following steps:
a) esterification reaction of a composition comprising an alcohol in the presence of a composition comprising lactic acid in excess,
b) extraction of a vapour phase from this reaction medium, containing lactic ester, alcohol, water and traces of lactic acid;
c) distillation of the vapour phase obtained at step b) in order to recover the lactic ester, alcohol and water at the top;
d) distillation of the fraction obtained at step c) in order to recover the purified lactic ester at the bottom.

2. Method according to claim 1, characterised in that the alcohol contains 1 to 12 carbons such as methanol, ethanol, n-butanol, isobutanol, sec-butanol, tert-butanol, n-propanol, isopropanol, 2-ethylhexanol, 2-ethylbutanol or hexanol.

3. Method according to claim 1, characterised in that the lactic acid composition has a concentration of acid of between 50% and 100% by weight in water.

4. Method according to claim 1, characterised in that the alcohol composition has an alcohol composition of between 30% and 100% by weight in water.

5. Method according to claim 1, characterised in that the esterification reaction is carried out at a pressure of 15 to 300 kPa absolute.

6. Method according to claim 1, characterised in that the esterification is performed at a temperature between 110° and 200° C.

7. Method according to claim 1, characterised in that the esterification reaction mixture has a lactic acid/alcohol mol ratio of between 1.1:1 and 50:1.

8. Method according to claim 1, characterised in that the esterification reaction is performed with a mol ratio of the alcohol/lactic acid mixture supplied continuously between 1:1 and 5:1.

9. Method according to claim 1, characterised in that the fraction extracted at the head of the first distillation column has an acidity of less than 0.5% by weight (for 100% lactic ester).

10. Method according to claim 1, characterised in that the purified lactic ester has a purity equal to or greater than 97%.
